(19) United States
(12) Patent Application Publication
(10) Pub. No.: US 2007/0264169 A1
(43) Pub. Date: Nov. 15, 2007

(54) Retaining clip for attaching a fragrance container to a cool air vent of an air conditioner

(76) Inventor: Wen-Jye Chen, Taipei (TW)

Correspondence Address: WEN-JYE CHEN;235 Chung - Ho, Box 8-24, Taipei, R.O.C., TW

(21) Appl. No.: 11/433,868

(22) Filed: May 15, 2006

(51) Int. Cl. A61L 9/12

(52) U.S. Cl. 422/124; 248/316.7

(57) ABSTRACT

A retaining clip for attaching a fragrance container to a cool air vent of an air conditioner comprises a retaining portion and a connecting portion integrally extended from the retaining portion. The retaining portion further comprises a pair of up and down arranged first and second clip arm. The first and the second clip arms are further provided with longitudinal slots. The free end of the first clip arm is tilted downward to form a pair of arced retaining sections. The free end of the second clip arm is provided with a pair of sloppy guide sections corresponding to the arced retaining section. At least one sloppy support bulge is formed on the middle section of the second clip arm. The free end of the connecting portion is connected to a fragrance container.

### FIELD OF THE INVENTION

The present invention relates to the art of attaching an object to the louvers across a cool air vent of an air conditioner, whereby a fragrance container will be stably mounted in front of the cool air vent and will be maintained horizontally for its proper operation.

### BACKGROUND OF THE INVENTION

When a driver starts up car air conditioner, a peculiar smell may produced inside the car, making people therein uncomfortable. Therefore, there are a variety of fragrance containers are used in front of a cool air vent to improve the car interior air quality. For example, the automobile fragrance container disclosed by R.O.C. patent number 408707, the improved fragrance container at an air vent disclosed by R.O.C. patent number 437393, and the improved fragrance container used in an automobile or in a room.

The fragrance containers of the prior art are either hung or clipped at the louvers across an air vent. In the former case, the container cannot be stably attached to the vent and may drop when the louvers are being adjusted. In the latter case, the clipping parts cannot be adapted to louvers of various thickness. Therefore, clipping parts corresponding various louvers have to be sold, or the container cannot be stably attached to the vent.

To summarize, the structures of the fragrance containers of the prior art are far from perfection. They cannot be stably mounted on a cool air vent. Further, when a user changes the inclination angle of the louvers across the vent, the container will tilt, possibly causing the spilling of the liquid fragrance therein.

### SUMMARY OF THE INVENTION

Accordingly, the primary objective of the present invention is to provide a retaining clip for attaching a fragrance container to a cool air vent of an air conditioner, wherein the fragrance container can be properly leveled so that the liquid fragrance therein will not spill.

To achieve the above objective, the present invention as a retaining clip for attaching a fragrance container to a cool air vent of an air conditioner comprises a retaining portion and a connecting portion integrally extended from the retaining portion.

The retaining portion further comprises a pair of up and down arranged first and second clip arm. The first and the second clip arms are further provided with longitudinal slots. The free end of the first clip arm is tilted downward to form a pair of arced retaining sections. The free end of the second clip arm is provided with a pair of sloppy guide sections corresponding to the arced retaining section. At least one sloppy support bulge is formed on the middle section of the second clip arm.

Further, the fragrance container is pivotally connected to the connecting portion by engaging its tongues between the ears of the connecting portion and then screwing a bolt and a nut along the axial hole of the engaged ears and tongues.

Thereby, the free end of the connecting portion is connected to a fragrance container, whereby a transverse louver across a cool air vent will go through the first clip arm and the second clip arm of the retaining portion. The retaining sections and the sloppy guide sections will engage with each other so that the retaining clip will be stably mounted on the air vent. Further, the inclination angle of the fragrance container can be adjusted so that the liquid fragrance in the container will not spill.

The various objects and advantages of the present invention will be more readily understood from the following detailed description when read in conjunction with the appended drawings.

### BRIEF DESCRIPTION OF THE DRAWINGS

FIG. 1 is a perspective view of a retaining clip for attaching a fragrance container to a cool air vent of an air conditioner of the present invention, which is connected with a fragrance container.

FIG. 2 is a side view of the retaining clip for attaching a fragrance container to a cool air vent of an air conditioner in FIG. 1, which is separated form the fragrance container.

FIG. 3 is a perspective view of the retaining clip for attaching a fragrance container to a cool air vent of an air conditioner in FIG. 1, which is engaged with a transverse louver across a cool air vent of an air conditioner.

FIG. 4 is a perspective view of the retaining clip for attaching a fragrance container to a cool air vent of an air conditioner in FIG. 1, which is engaged with a vertical louver across a cool air vent of an air conditioner.

### DETAILED DESCRIPTION OF THE PREFERRED EMBODIMENTS

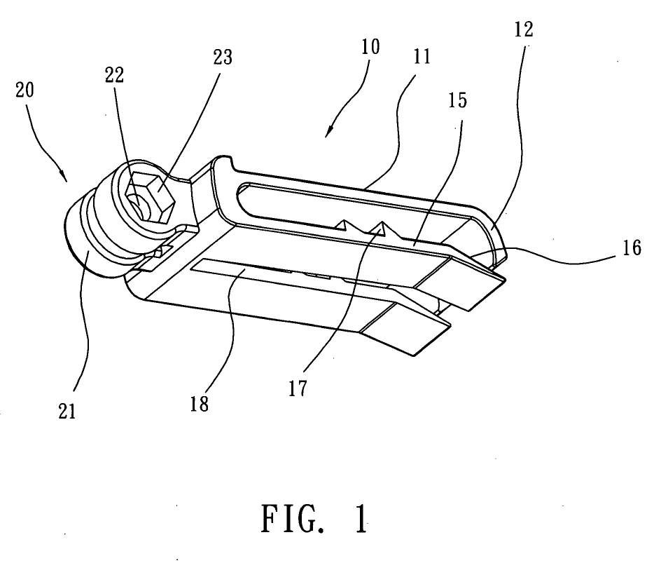

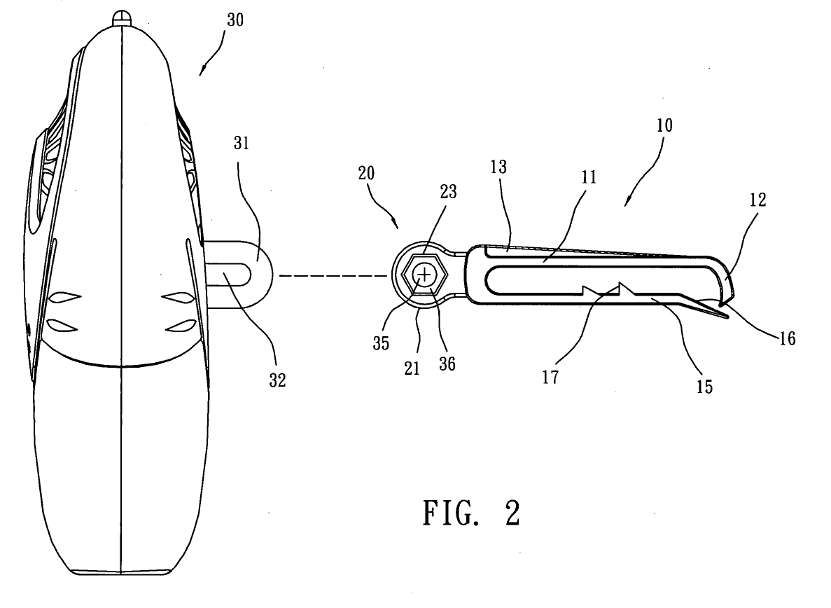

Referring to FIGS. 1 and 2, a retaining clip for attaching a fragrance container to a cool air vent of an air conditioner comprises a retaining portion 10 capable of being mounted on a louver 51 across a cool air vent 50 of an air conditioner and a connecting portion 20 connected with a fragrance container 30.

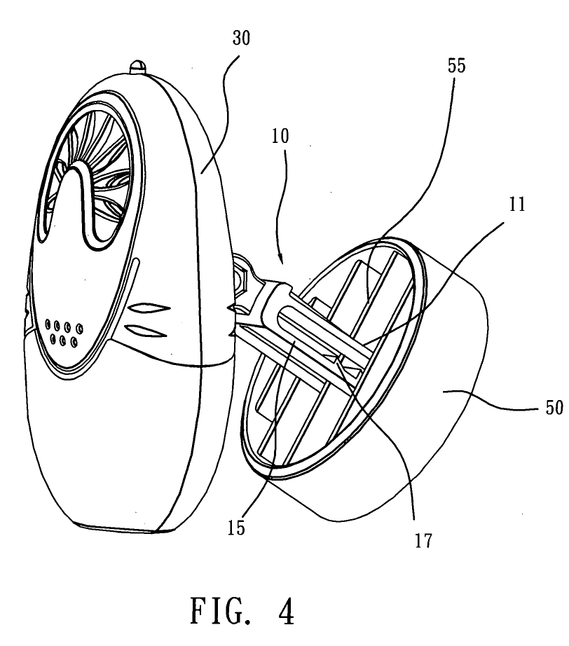

As shown in FIGS. 1, 2 and 3, the retaining portion 10 of the retaining clip further includes a pair of opposite first clip arm 11 and second clip arm 15, which form an integral portion at one end. The other end of the first clip arm 11 forms an inwardly arced section 12 and a sloppy top reinforcing strip 13 for not only enhancing the structural toughness of the first clip arm 11 but also the pressing of the upper transverse neighboring louver 51. The second clip arm 15 has a sloppy guide section 16 corresponding to the arced section 12 for pinching the rear edge along an engaged transverse louver 51. The inner wall of the second clip arm 15 further includes a pair of right and left support bulges 17 for pressing the engaged transverse louver 51. Further, each of the first clip arm 11 and the second clip arm 15 has a longitudinal slot 18 for clipping a vertical louver 55, as shown in FIG. 4.

The connecting portion 20 is an integral part of and extended from the closed end of the retaining portion 10, which has a pair of parallel ears 21. Each of the ears 21 has a central through hole 22, whose terminal on the outer face of a corresponding one of the ears 21 forms a polygonal terminal depression 23. Between the ears 21, a fragrance container 30 is retained by the engagement of the ears 21 and two tongues 31 thereon. Each of the tongues 31 further includes a through hole 32 aligned with a corresponding one of the through holes 22, whereby a bolt 35 and a nut 36 will go through the through holes 22 and 32 for securing the fragrance container 30. Further, the fragrance container 30 can rotate about the connecting portion 20, and the resistant damping therebetween can secure the fragrance container 30 to an adjustable inclination angle.

Thereby, a retaining clip capable of holding a fragrance container stably and adjusting the fragrance container to a proper inclination angle, prohibiting the outflow of fragrance liquid, is achieved.

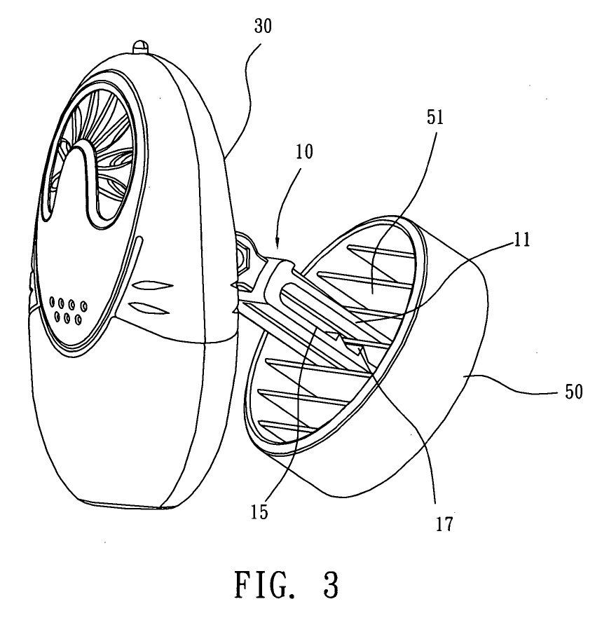

To use the present invention, FIGS. 1 and 3 illustrate the first clip arm 11 and the second clip arm 15 engage with a transverse louver 51 of a cool air vent 50. The ends of the first clip arm 11 and the second clip arm 15 are aimed at a transverse louver 51, whereby the transverse louver 51 is inserted into the space between the first clip arm 11 and the second clip arm 15. The reinforcing strip 13 on top of the first clip arm 11 will be pressed downward by the transverse louver 51, whereby the support bulges 17 on the second clip arm 15 will be supported against the clipped second clip arm 15. Therefore, the retaining clip is stably mounted on the cool air vent 50. Further, the arced section 12 will be engaged with the rear edge of the transverse louver 51 for preventing the clip from falling off. Further, since the width of the first clip arm 11 and the second clip arm 15 is larger than the width of the vertical louvers 55, the slots 18 along the first clip arm 11 and the insulating piece 12 will erase the interference of a vertical louver 55 as the clip is inserted to clip a transverse louver 51.

Further, as shown in FIGS. 1 and 4, if a cool air vent 50 has vertical louvers 55 arranged in front of transverse louvers 51, the slots 18 along the first clip arm 11 and the insulating piece 12 will dodge the interference of one of the vertical louvers 55 first as the clip is inserted to clip a transverse louver 51 behind. The ends of the first clip arm 11 and the second clip arm 15 are aimed at a transverse louver 51, whereby the transverse louver 51 is inserted into the space between the first clip arm 11 and the second clip arm 15. The reinforcing strip 13 on top of the first clip arm 11 will be pressed downward by the transverse louver 51, whereby the support bulges 17 on the second clip arm 15 will be supported against the clipped second clip arm 15. Therefore, the retaining clip is stably mounted on the cool air vent 50. Further, the arced section 12 will be engaged with the rear edge of the transverse louver 51 for preventing the clip from falling off.

Further, the fragrance container 30 is pivotally connected to the connecting portion 20 by engaging its tongues 31 between the ears 21 and then screwing a bolt 35 and a nut 36 along the axial hole of the ears 21 and the tongues 31, as shown in FIGS. 2, 3 and 4. As the bolt 35 and the nut 36 are fastened, the fragrance container 30 can be secured at a preferred inclination angle. Further, the angle of the fragrance container 30 can be freely adjusted even when it is mounted onto the air vent 50. Therefore, the fragrance liquid within the fragrance container 30 will not spill.

The present invention is thus described, and it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A retaining clip for attaching a fragrance container to a cool air vent of an air conditioner, comprising:
a retaining portion further comprising a pair of up and down arranged first and second clip arm, a free end of said first clip arm being tilted downward to form a pair of arced retaining sections, a free end of said second clip arm being provided with a pair of sloppy guide sections corresponding to said arced retaining section, at least one sloppy support bulge being formed in a middle section of said second clip arm; and
a connecting portion extended from said retaining portion having a pair of parallel ear parts for engaging a corresponding pair of tongues extended from a fragrance container, each of said ear parts and each of said tongues having an axial through hole, whereby said fragrance container and connecting portion will be connected by screwing a bolt and a nut along the coupled axial through holes of said ear parts and said tongues;
whereby said fragrance container will be stably connected to said retaining clip, and whereby the inclination angle of said fragrance container with respect to said cool air vent can be adjusted so that liquid fragrance in said fragrance container will not spill

2. The retaining clip for attaching a fragrance container to a cool air vent of an air conditioner of claim 1 wherein a top surface of said first clip arm of said retaining portion is provided with a sloppy reinforcing strip descended toward said free end of said first clip arm.

3. The retaining clip for attaching a fragrance container to a cool air vent of an air conditioner of claim 1 wherein central axial portions of said first clip arm and said second clip arm are respectively provided with longitudinal slots for avoiding the interference of a vertical louver when said retaining clip is clipping a transverse louver.

4. The retaining clip for attaching a fragrance container to a cool air vent of an air conditioner of claim 1 wherein each of said ear parts of said connecting portion has an outer polygonal depression around an entrance of a corresponding one of said through holes for housing said nut when said nut is fastened on said bolt.
